(19) United States
(12) Patent Application Publication
(10) Pub. No.: US 2007/0286836 A1
(43) Pub. Date: Dec. 13, 2007

(54) Removal of hair cosmetics from show animals

(76) Inventors: Leonard Hughes, Dublin, OH (US); Steven L. Funk, Grove City, OH (US)

Correspondence Address: MUELLER AND SMITH, LPA;MUELLER-SMITH BUILDING, 7700 RIVERS EDGE DRIVE, COLUMBUS, OH, 43235, US

(21) Appl. No.: 11/811,188

(22) Filed: Jun. 8, 2007

Related U.S. Application Data
(60) Provisional application No. 60/812,891, filed on Jun. 12, 2006.

(51) Int. Cl. A61K 8/55; A61K 8/37

(52) U.S. Cl. 424/70.23; 424/70.31

(57) ABSTRACT

The formulation disclosed comprises of a suitable solvent at a level for removing the hair stiffeners and other hair-adhered chemicals (say, from about 70% to about 95% w/w); an emulsifier at a level effective to emulsify the removed chemicals (say, from about 5% to about 25% w/w); and, optionally, a hair conditioner for the animal's hair (say, from about 0.5 to about 5% w/w). One embodiment is a two-part formulation having 85% w/w soy methyl ester and 15% w/w of a phosphate ester emulsifier. Both ingredients are readily biodegradable. A hair conditioner can be included in the formulation to leave the hair softer. The formulation also may be pre-emulsified.

### CROSS-REFERENCE TO RELATED APPLICATIONS

This application claims benefit of provisional application Ser. No. 60/812,891, filed on Jun. 12, 2006, the disclosure of which is expressly incorporated herein by reference.

### STATEMENT REGARDING FEDERALLY SPONSORED RESEARCH

Not applicable.

### BACKGROUND

The present disclosure generally relates to removal of hair stiffeners and more particularly to a new composition for removal of hair cosmetics from show animals.

In the showing of cattle at various professional functions throughout the USA, and indeed throughout the world, considerable use is made of a range of cosmetics to enhance the animals' appearance. In cattle showing, for example, the show cattle are led before the judges in the show arena at a slow pace or kept stationary in front of the judges. As with any show animal, there are certain parameters that the judges consider when evaluating the cattle, one of these being the overall body profile and appearance. In preparation for this, considerable grooming effort is made prior to the showing. Even though the whole body is cleaned and treated with cosmetics, great attention is given to the legs and the tail. This involves the use of various hair treatment formulations, which have to be removed after the show.

Traditionally, the treated legs are sprayed with a hydrocarbon-based remover currently dominating the market, which cuts into the resins used in the applied cosmetics. After partly dissolving the cosmetics, an air hose is used to blow off the resulting fluid sludge from the legs. This is the step that causes an air pollution hazard in the barn as it removes the majority of the chemical residues. It also is what could cause color cross contamination from a “blown” animal to a neighboring animal under preparation for showing. Air pressure is used extensively in show cattle dressing, and subsequent removal of the cosmetics after the showing. Following this step the legs have to be washed with a shampoo to remove the last traces of the cosmetics. If the blowing step is omitted, the removal of the cosmetics using considerable hand labor is left to the shampoo alone, and the demand of the shampoo is increased considerably. The blowing step currently dominates the removal process.

The present disclosure is directed to improvements in this removal process and to elimination of air pollution caused by current commercial practice.

### BRIEF SUMMARY

The formulation disclosed comprises of a suitable solvent at a level for removing the hair stiffeners and other hair-adhered chemicals (say, from about 70% to about 95% w/w); an emulsifier at a level effective to emulsify the removed chemicals (say, from about 5% to about 25% w/w); and, optionally, a hair conditioner for the animal's hair (say, from about 0.5 to about 5% w/w). One embodiment is a two-part formulation having 85% w/w soy methyl ester and 15% w/w of a phosphate ester emulsifier. Both ingredients are readily biodegradable. A hair conditioner can be included in the formulation to leave the hair softer. The formulation also may be pre-emulsified. For present purposes, “w/w” means weight-by-weight basis.

Advantages of the present remover include the removal of hair stiffeners and cosmetics quickly. Another advantage is the absence of air blowing in the disclosed process. A further advantage is the use of biodegradable products in the disclosed hair chemical remover. These and other advantages will be readily apparent to those skilled in the art based on the disclosure set forth herein.

### DETAILED DESCRIPTION

This is What is Done to the Legs in Preparation for the Show:

A polymeric hair stiffener is applied to the leg hair from an aerosol can (or a spray bottle) whilst combing the hair to make it stand out parallel to the ground (i.e., at right angles to the leg). This product is known in the cattle showing business as an “adhesive”. The stiffening happens when the product has dried (fairly rapidly).

Certain paints then are sprayed onto the stiffened hair for the purpose of “filling” (white) and, when allowed by the state regulation, black, brown, or red-brown colors. These colors are used to color match the treated legs with the upper body of the animal.

The hair is trimmed with electric clippers to obtain the desired leg profile. The net effect is to realize a plumped up or fuller look in the legs. The stiffener is used on the tail, particularly the base of the tail, to also raise the hair artificially. For convenience, animal hair stiffeners, paints, and like chemicals will be termed “animal cosmetics” for purposes of the disclosed remover.

After the Showing:

After the showing, the accumulated cosmetics have to be removed; either at the show barn or after the cattle have been returned to the farm. The chemical residues from the adhesive include most likely a hard resin of some kind, resin binders in the paint formulations, the pigments or dyes residual from the colorants, and any other ingredients typically found in such preparations. As described above, traditional removers are air sprayed off of the animal to create airborne pollution and contamination of nearby animals, among other problems.

Disclosed Hair Cosmetics Remover

The subject of this application is a liquid formulation of a suitable solvent containing any emulsifier capable of emulsifying the solvent and the dissolved cosmetics. The purpose of the cosmetics removal formulation is to treat the animal legs and tail (or the body, if required) with a spray on-rinse off cosmetics remover, which can be rinsed off with water alone and avoid the blowing and shampooing steps. By suitable solvent we mean a solvent capable of dissolving the cosmetic components. Any emulsifier capable of producing subsequent emulsification with the rinse water may be used, including, for example, one or more of fatty acid soaps, alcohol ethoxylates, alkanolamides, or fatty acid ethoxylates. The emulsion does not have to be a permanently, stable one, but sufficiently transient to perform the intended use. Fatty acids, for present purposes are C₆ to C₂₂ acids typical of vegetable oils, tall oils, and the like. Additional emulsifiers can be found in, for example, McCutcheon's Detergents & Emulsifiers. (1973) (Glen Rock, N.J.: McCutcheon Division, MC Pub. Co.), the disclosure of which is expressly incorporated herein by reference.

The emulsifier can be taken from all the classes of anionic, nonionic, and cationic emulsifiers, as the only requirement is to be able to emulsify the solvent and removed cosmetics. There may be additional benefits to using emulsifiers, which can impart a conditioning effect to the animal hair. There is no reason why more than one non-solvent component cannot be utilized to achieve greater hair conditioning.

With regard to solvent selection, there are environmental benefits to selecting a readily biodegradable solvent. The subject formulation has the additional benefit of being able to dissolve residues on water-wet hair, currently not achievable with existing removers. For example, if the person grooming the animal misses a spot with the spray, he can apply the product subsequently to the wet hair and still achieve residue removal. Suitable solvents include, then, one or more of hydrocarbons, alcohols, esters, light mineral oils, ethers, glycol ethers, ketones, or combinations of such solvents.

The disclosed formulation and process results in a decrease time of removal of residues through a “Spray on-Rinse off” process; decreased air pollution hazards with all residues going to a waste water plant. Of course, any animal hair can be treated in accordance with the disclosed remover. Additionally, the disclosed remover results in a considerable decrease in the time required for the removal of the cosmetics, as current procedures of application and removal are very labor intensive; and a reduction of air pollution in the communal barn wherein all contestants treat their animals. The hair is left in a soft and pleasant condition with no adverse after effects, such as, for example, stiffness, artificial color or (skin) irritation. All cosmetic residues are directed to the wastewater drain in the barn and, thus, subjected to controlled biodegradation in the municipal water treatment plant rather than put into the atmosphere to be inhaled by the animal attendants and others in the barn area. There also is no cross-contamination by cosmetic residues between cattle through air pollution. The disclosed remover system also has the capability of treating water-wet hair and still achieve residue removal with no shampooing being necessary.

While the formulation and method have been described with reference to various embodiments, those skilled in the art will understand that various changes may be made and equivalents may be substituted for elements thereof without departing from the scope and essence of the disclosure. In addition, many modifications may be made to adapt a particular situation or material to the teachings of the disclosure without departing from the essential scope thereof. Therefore, it is intended that the formulation and method not be limited to the particular embodiments disclosed, but that the formulation and method will include all embodiments falling within the scope of the appended claims. In this application all amounts and percentages are by weight, unless otherwise expressly indicated. Also, all citations referred herein are expressly incorporated herein by reference.

I claim:

1. A formulation for removing animal cosmetics from the hair of an animal, which comprises:
(a) a solvent in an amount effective to solvate the animal cosmetics; and
(b) an emulsifier in an amount effective to emulsify the animal cosmetics solvated by said solvent.

2. The formulation of claim 1, wherein said solvent is present in amount of between about 70% and about 95% w/w.

3. The formulation of claim 1, wherein said solvent is one or more of a hydrocarbon, an alcohol, an ester, a light mineral oil, an ether, a glycol ether, or a ketone.

4. The formulation of claim 3, wherein said solvent comprises soy methyl ester.

5. The formulation of claim 1, wherein said emulsifier is one or more of an anionic emulsifier, a cationic emulsifier, or a non-ionic emulsifier.

6. The formulation of claim 5, wherein said emulsifier is one or more of a fatty acid soap, an alcohol ethoxylate, an alkanolamide, or a fatty acid ethoxylate.

7. The formulation of claim 6, wherein said emulsifier comprises a phosphate ester emulsifier.

8. The formulation of claim 1, wherein said emulsifier is present in an amount of between about 5% and about 25% w/w.

9. The formulation of claim 1, which additionally comprises a hair conditioner.

10. The formulation of claim 8, wherein said hair conditioner is present in an amount of between about 0.5% to about 5% w/w.

11. The formulation of claim 1, wherein said solvent comprises about 85 wt-% soy methyl ester, about 15 wt-% phosphate ester emulsifier.

12. A method for removing animal cosmetics from the hair of an animal, which comprises the steps of:
(a) applying a formulation to said animal hair, said formulation comprising a solvent in an amount effective to solvate the animal cosmetics; and
an emulsifier in an amount effective to emulsify the animal cosmetics solvated by said solvent; and
(b) rinsing said animal hair to remove said solvent and said solvated animal cosmetics.

13. The method of claim 12, wherein the solvent in the formulation applied to said animal hair is present in amount of between about 70% and about 95% w/w.

14. The method of claim 13, wherein the solvent in the formulation applied to said animal hair is one or more of a hydrocarbon, an alcohol, an ester, a light mineral oil, an ether, a glycol ether, or a ketone.

15. The method of claim 12, wherein the emulsifier in the formulation applied to the animal hair is one or more of an anionic emulsifier, a cationic emulsifier, or a non-ionic emulsifier.

16. The method of claim 15, wherein the emulsifier in the formulation applied to the animal hair is one or more of a fatty acid soap, an alcohol ethoxylate, an alkanolamide, or a fatty acid ethoxylate.

17. The method of claim 16, wherein the emulsifier in the formulation applied to the animal hair is present in an amount of between about 5% and about 25% w/w.

18. The method of claim 1, wherein the formulation applied to the animal hair additionally comprises a hair conditioner.

19. The method of claim 8, wherein the hair conditioner in the formulation applied to the animal hair is present in an amount of between about 0.5% to about 5% w/w.

20. The method of claim 1, wherein the formulation applied to the animal hair comprises about 85 wt-% soy methyl ester solvent and about 15 wt-% phosphate ester emulsifier.
